(19) United States
(12) Patent Application Publication
(10) Pub. No.: US 2018/0221371 A1
(43) Pub. Date: Aug. 9, 2018

(54) TREATMENT FOR THE FETUS WITH CONGENITAL HEART DISEASE

(71) Applicant: CHILDREN'S NATIONAL MEDICAL CENTER, Washington, DC (US)

(72) Inventors: Nobuyuki ISHIBASHI, Washington, DC (US); Richard A. JONAS, Washington, DC (US)

(73) Assignee: CHILDREN'S NATIONAL MEDICAL CENTER, Washington, DC (US)

(21) Appl. No.: 15/749,792

(22) PCT Filed: Aug. 8, 2016
(86) PCT No.: PCT/US2016/046016

§ 371 Date: Feb. 2, 2018

Related U.S. Application Data
(60) Provisional application No. 62/202,336, filed on Aug. 7, 2015.

(51) Int. Cl. A61K 31/519; A61K 9/00; A61P 9/00

(52) U.S. Cl. CPC A61K 31/519; A61K 9/0034; A61K 31/198; A61K 2300/00; A61P 9/00

(57) ABSTRACT

A method for treating injury or insult to the brain or nervous system caused by or associated with hypoxia, especially hypoxia associated with injury or insult to white matter in the foetal brain of a subject having congenital heart disease, by administering BH4.

### CROSS-REFERENCE TO RELATED APPLICATIONS

This application claims benefit of U.S. Provisional Application No. 62/202,336, filed Aug. 7, 2015 which is hereby incorporated by reference in its entirety.

### STATEMENT REGARDING FEDERALLY SPONSORED RESEARCH OR DEVELOPMENT

This invention was made with government support under National Institute of Health grant NIH R01HL104173. The government may have certain rights in the invention.

### BACKGROUND OF THE INVENTION

### Field of the Invention

Prevention, amelioration or treatment of hypoxic injury or insult to the brain or nervous system of a fetus resulting from congenital heart defect or disease (“CHD”) by administering a cofactor of neuronal nitric acid synthase (“nNOS”) tetrahydrobiopterin (“BH4”). Hypoxic injury and its subsequent effects persist and can present life-long challenges for children who have or have had CHD, especially severe or complex CHD.

### Description of Related Art

CHD is the most common major birth defect^{1, 2}; worldwide, approximately 3 million infants are born with CHD each year with approximately 25% of cases comprising severe/complex CHD^{3, 4}. Over the last 2 decades amazing advances have been made in reducing the mortality risk for patients with severe/complex CHD from close to 100% to less than 10%⁵. However, many of these children later suffer significant neurological impairment and behavioral problems that follow from CHD⁶⁻¹². As a consequence of improved survival, the prevalence of severe/complex CHD is 1.8 per 1,000 children in the North America and this population is steadily rising with reports describing a 19% increase in children and 55% in adults^{13, 14}. Prolonged neurological deficits pose substantial socioeconomic and management challenges for patients, families, and society. Reducing CHD-induced neurological deficits remains not only a fundamental research endeavor: it is vital for the healthcare of this growing community of patients¹⁵.

Abnormal white matter (“WM”) development accounts for neurological deficits seen in patients with CHD. Worse outcomes for motor skills than cognitive abilities have been well documented in CHD patients 1-4 years of age after surgery¹⁶⁻¹⁸. These findings are consistent with WM injury¹⁹. School-age children with complex CHD exhibit motor, cognitive, and behavioral difficulties encompassing problems in a broad range of skills: visual-spatial, fine/gross motor, memory, attention, language, executive, and psychosocial^{20, 21}. These developmental challenges are remarkably similar to the deficits observed in preterm survivors in whom WM injury is a major source of morbidity^{22, 23}. Adolescents with d-TGA performed lower than the population mean on academic achievement, memory, visual-spatial skills, social cognition, and attention^{24, 25}. Recent studies displayed altered WM microstructure in these adolescents²⁶⁻²⁸. It is well known that in premature birth, alterations of the WM microstructure persist into later life²⁹. The extent of abnormal WM development early in life likely accounts for the type and degree of neurological deficits observed in patients with CHD.

Many CHD anomalies are associated with immature brain at birth due to reduced oxygen supply in utero. Within multifactorial etiologies of neurological deficits in CHD^{10, 11}, sophisticated imaging techniques are bringing prenatal events associated with neurological injury into focus^{11, 30}. Fetal cerebral blood flow involves preferential streaming of the most highly oxygenated blood to the developing brain. However, severe/complex CHD—e.g., d-TGA or HLHS—can alter these beneficial patterns, resulting in delivery of de-saturated blood to the brain^{31, 32, 30}. Because the heart is nearly fully developed by the 7th week of gestation, CHD can disrupt fetal cerebral oxygen delivery for more than 7 months during a period critical for brain development. Studies have demonstrated delayed brain maturation and WM immaturity at birth in CHD^{31, 33, 34-36}. Further WM injury also commonly occurs after surgery in these same individuals who have WM immaturity due to fetal hypoxia^{37, 38}. To reduce neurological deficits in CHD patients, therefore, it will be necessary to mitigate chronic hypoxia-induced immature WM development in the fetus with CHD. However, no treatment options are currently available.

Significant neurodevelopmental delay is emerging as one the most important current challenges for patients with congenital heart disease (CHD)^{20, 21}. Abnormal white matter development early in life accounts for the type/degree of neurological deficits observed in children with CHD^{31, 39}. In these children, WM is immature at birth due to reduced oxygen supply in utero^{32, 33}. Further WM injury after cardiac surgery also commonly occurs in these same individuals who have WM immaturity due to fetal hypoxia^{37, 38}. BH4 has been used as an early treatment for children with phenylketonuria and positive effects on white matter where observed^{40, 41}. However, neither BH4 treatment nor any other good treatment options for treating WM injury or immature WM development associated with CHD in utero exist. Consequently, the inventors studied the effects of CHD on the developing brain with the objective of finding a treatment that would ameliorate the detrimental effects of CHD on white matter and brain development in a fetus with CHD and to reduce white matter injury in the fetus after cardiac surgery for CHD.

### BRIEF SUMMARY OF THE INVENTION

One aspect of the invention is a method for treating a subject who has, or who is at risk of having hypoxia, especially a subject in utero who has complex congenital heart disease, by administering BH4. The inventors have found that there are decreased levels of BH4 in the hypoxic brain, a condition that occurs in complex CHD, and, surprisingly, that treating with BH4 mitigates the toxic effects of hypoxia in a murine model of hypoxia associated with CHD.

### BRIEF DESCRIPTION OF THE SEVERAL VIEWS OF THE DRAWING

FIG. 1. Glutamate transporter failure causes Ca²⁺ flooding, which increases nNOS activity. This causes calcium flooding which leads to increased neuronal NOS activation^{42, 43, 44, 45}.

FIG. 2. Comparison of H4 Biopterin (BH4) coupled and uncoupled reactions. When the cofactor, BH4, is absent, the uncoupled NOS reaction produces reactive oxygen species instead of water. Absence of tetrahydrobiopterin (BH4) cofactor during the formation of NO leads to production of reactive oxygen species⁴⁶.

FIG. 3. Correspondence between human and rodent gestation periods. Regarding WM development, postnatal days 3-11 in mice correspond to the 3^{rd} trimester of human pregnancy. The murine model was used by the inventors to investigate the effects of hypoxia and treatment of hypoxia by BH4. A mouse model of chronic hypoxia during a period that corresponds to the 3^{rd} trimester in human white matter development is described by Scafidi, et al ⁴⁷ and by Raymond, et al. ^{48,} both of which are incorporated by reference.

FIG. 4. Immunohistochemistry images of WM tracts. Green (light areas) cells positive for CNP, thus represent oligodendrocytes. Hypoxia led to increased oligodendrocyte density. This effect was not limited by BH4 therapy.

FIG. 5. Density of oligodendrocytes (CNP positive cells). As shown, hypoxia led to increased oligodendrocyte density in Hx and Hx-BH4 groups compared to the normal control (Nx). This proliferative effect was not limited by BH4 therapy.

FIG. 6. Immunohistochemistry of white matter (“WM”) tracts. Yellow cells (center pane, boxed) were positive for both CNP and Caspase3, representing apoptotic oligodendrocytes. Apoptosis was increased in hypoxia. BH4 therapy was found to mitigate this effect.

FIG. 7. Density of apoptotic cells (Caspase3 positive). Hypoxia caused increased apoptosis as shown by a comparison between the normal control (Nx) and the hypoxic (Hx) group. BH4 therapy was found to mitigate this effect as shown by the Hx-BH4 group.

FIG. 8. Immunohistochemistry images of WM tracts. Myelin basic protein is stained in red (lighter gray) and demonstrates the amount of myelination present. Decreased myelination was seen in hypoxic group (center pane, Hx) compared to control (Nx). These effects were mitigated by BH4 treatment (right pane, Hx-BH4).

FIG. 9. Expression of Myelin Basic Protein. Decreased myelination compared to normal control (Nx) was seen in hypoxic group (Hx). Decreased myelination was found to be mitigated BH4 treatment such that there was no significant difference between normoxic controls (Nx) and hypoxic mice treated with BH4 (Hx-BH4).

FIG. 10. Western blot for expression of Myelin Basic Protein. Loss of myelination in hypoxia (Hx) was found to be mitigated by BH4 therapy (Hx-BH4).

FIG. 11. Brain BH4 levels (left bar) were depleted by 38.4% in hypoxia (right bar).

### DETAILED DESCRIPTION OF THE INVENTION

A method for treating a subject who is at risk of, or who is undergoing hypoxic injury to white matter in the brain or nervous system by administering BH4, especially treatment of a subject in utero undergoing hypoxic injury to white matter in the brain due to congenital heart defect or complex congenital heart defect. The inventors investigated whether there were decreased levels of BH4 in the hypoxic brain, a condition that occurs in complex congenital heart defect (“CHD”). Surprisingly, it was found that the toxic effects of hypoxia on the brain could be mitigated by the administration of BH4.

BH4: Tetrahydrobiopterin, sapropterin or H4 Biopterin is a naturally occurring essential cofactor of the three aromatic amino acid hydroxylase enzymes, used in the degradation of amino acid phenylalanine and in the biosynthesis of the neurotransmitters serotonin (5-hydroxytryptamine, 5-HT), melatonin, dopamine, norepinephrine (noradrenaline), epinephrine (adrenaline), and is a cofactor for the production of nitric oxide (NO) by the nitric oxide synthases. BH4 is identified by CAS Registry Number: 17528-72-2.

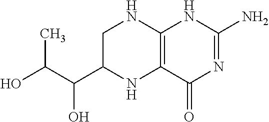 [SMILES: CC(O)C(O)C1CNc2[nH]c(N)nc(=O)c2N1]

As defined herein, this term includes salts, solvates, polymorphs, and amorphous forms of BH4.

A synthetic formulation of BH4 (sapropterin dihydrochloride) was approved by the FDA in 2007 for phenylketonuria⁴⁹. There have been extensive safety trials for the implementation of BH4 therapy in humans⁵⁰. From long-term follow-up studies, BH4 was found to be safe and well tolerated at a wide range of doses^{51, 52}. Since BH4 is a natural co-factor and no teratological effects have been documented⁵³, BH4 has been approved for use in pregnancy. Recent trials have concluded that maternal BH4 treatment is efficient and safe in pregnant females⁵⁴⁻⁵⁶. BH4 crosses the placenta and distributes throughout fetal tissues, including the brain^{88, 89}. Improvements of WM integrity and damage were found in children with phenylketonuria treated early with BH4^{40, 41}. The administration modes, frequency of administration, excipients and dosages described by the references cited above are hereby incorporated by reference.

BH4 may be in an amorphous form, a polymorph, solvate or hydrate or other form suitable for administration to a subject or for formulating a composition suitable for administration to a subject. Suitable pharmaceutically acceptable carriers for BH4 include but are not limited to water, salt solutions (e.g., normal NaCl/saline), buffered saline, alcohols, glycerol, ethanol, gum arabic, vegetable oils, benzyl alcohols, polyethylene glycols, gelatin, carbohydrates such as lactose, amylase or starch, dextrose, magnesium stearate, talc, silicic acid, viscous paraffin, fatty acid esters, hydroxymethylcellulose, polyvinyl pyrrolidone, etc., as well as combinations thereof. In addition, carriers such as liposomes and microemulsions may be used. BH4 or its analogs may be bound to a protein carrier such as albumin, or a polymer, such as polyethylene glycol so as to modulate pharmacokinetics such as to prolong its biological half-life or facilitate it crossing the placenta or blood-brain barrier and reaching the foetal brain. Auxiliary agents, such as lubricants, preservatives, stabilizers, wetting agents, emulsifiers, salts for influencing osmotic pressure, buffers, coloring, flavoring and/or aromatic substances and the like that do not deleteriously react with BH4 or its analogs may be included.

A therapeutically effective amount of BH4 for the treatment of a particular patient having a disorder or condition will depend on the nature of the disorder or condition, and can be determined by standard clinical techniques. In vitro or in vivo assays may be employed to identify optimal dosage ranges. The precise dose to be employed in the formulation will often depend on the route of administration and the severity of the symptoms of the disease or condition, and should be decided according to the judgment of a practitioner and each patient's circumstances. Effective doses may be extrapolated from dose-response curves derived from in vitro or animal model test systems. One example of a BH4 dosage for oral administration to a pregnant woman or other subject is 50 mg/kg/day. Other dosages may be administered orally or by other modes of administration and include >0, 0.5, 1, 2, 3, 4, 5, 6, 7, 8, 9, 10, 20, 30, 40, 50, 60, 70, 80, 90, 100, 150, 200, 250, 300, 350, 400, 450, 500 or more mg/kg/day as well as intermediate subranges and values.

Other active agents or excipients may be administered in conjunction with treatment with BH4, for example, before, at the same time, or after BH4 administration. These include arginine, which is a substrate for nNOS, antioxidants, such as ascorbate, tocopherol, glutathione, cysteine, acetyl-cysteine, etc., or inhibitors of nNOS. Other excipients include those that facilitate passage of BH4 from the site of administration into the foetus or across the blood-brain barrier of the subject undergoing treatment.

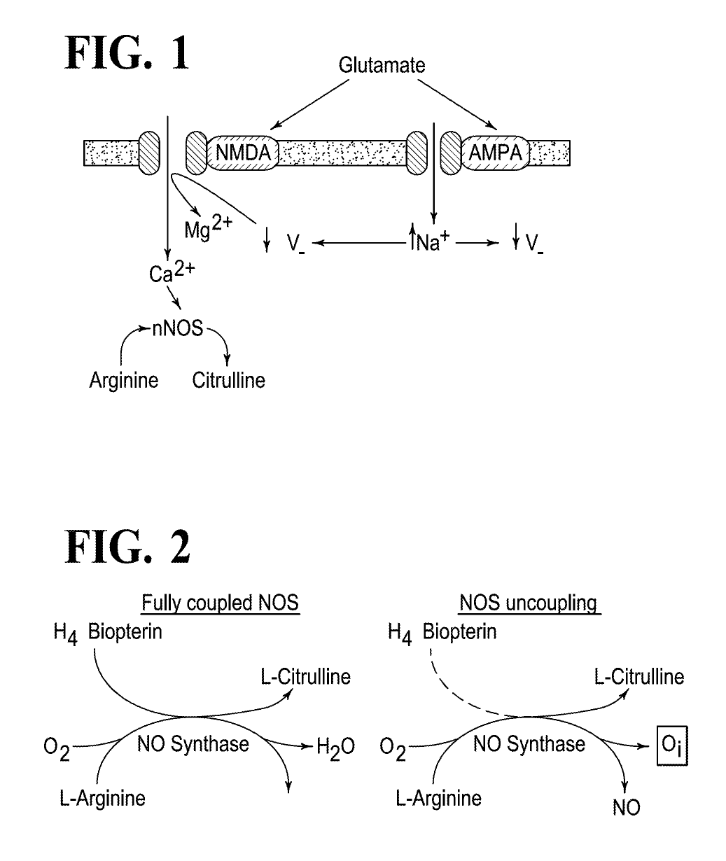

CHD: Congenital heart defect. Congenital heart defects are known by a number of names including congenital heart defect, complex congenital heart defect, complex congenital heart defect at birth, congenital cardiovascular malformations, congenital heart anomaly, and congenital heart disease. A congenital heart defect is a problem with the structure of the heart. It develops in utero and generally is present at birth. Congenital heart defects are the most common type of major birth defect. A baby's heart begins to develop shortly after conception. During development, structural defects can occur. These defects can involve the walls of the heart, the valves of the heart and the arteries and veins near the heart. Congenital heart defects can disrupt the normal flow of blood through the heart. The blood flow can slow down, go in the wrong direction or to the wrong place, or be blocked completely. Altered patterns of circulation n complex congenital heart disease (CHD) lead to reduced or desaturated cerebral blood flow in utero^{20, 21, 31}. There is a high incidence of white matter (WM) injury (25-55%) in the infant with complex CHD^{32, 33, 37, 39}. The first event in brain hypoxia is glutamate transporter failure^{38, 57}. This causes calcium flooding which leads to increased neuronal NOS activation^{58, 59} (FIG. 1). Absence of tetrahydrobiopterin (BH4) cofactor during the formation of NO leads to production of reactive oxygen species⁶⁰ (FIG. 2). Subjects having CHD as well as the particular physiological conditions described above may be treated by the methods of the invention.

Congenital malformations of the circulatory system. These include congenital heart defects as well as malformations of other parts of the circulatory system such as arteries, veins, or circulatory system valves, including those that affect the transport of blood to and from the brain, lungs or heart. Subjects having such defects may be treated by the methods described herein.

Glial cells. These include oligodendrocytes, astrocytes, ependymal cells and microglia. These cells surround neurons and hold them in place, facilitate or assist neurons in forming synaptic connections between each other, supply nutrients and oxygen to neurons, insulate one neuron from another, and destroy pathogens and remove dead neurons. The methods described herein may modulate or ameliorate damage to glial cells as well as to other cells of the nervous system such as neurons.

Hypoxia. A deficiency in the amount of oxygen reaching the tissues. The methods described herein ameliorate the detrimental effects of hypoxia that can affect the growth and development of the brain. The inventors recognized that white matter injury and immaturities in a fetus, even after cardiac surgery to correct CHD, is associated with hypoxia and have now found that the administration of BH4 ameliorates these detrimental effects.

Modes of administering BH4 or the other compounds described are known and include, but are not limited to, oral administration and parenteral administration. Parenteral administration includes, but is not limited to intradermal, intramuscular, intraperitoneal, intravenous, subcutaneous, intranasal, epidural, sublingual, intranasal, intracerebral, intraventricular, intrathecal, intravaginal, transdermal, rectal, by inhalation, or topically to the ears, nose, eyes, or skin. Other methods of administration include but are not limited to infusion techniques including infusion or bolus injection, by absorption through epithelial or mucocutaneous linings such as oral mucosa, rectal and intestinal mucosa. Compositions for parenteral administration may be enclosed in ampoule, a disposable syringe or a multiple-dose vial made of glass, plastic or other material.

Administration may be systemic or local. Systemic administration includes oral or intravenous administration to a subject. Local administration is administration of the disclosed compound to the area in need of treatment. Examples include local infusion during surgery; topical application, by local injection; by a catheter; by a suppository; or by an implant. Administration may be by direct injection into the central nervous system by any suitable route, including intraventricular and intrathecal injection. Intraventricular injection can be facilitated by an intraventricular catheter, for example, attached to a reservoir, such as an Ommaya reservoir. Pulmonary administration may be achieved by any of a number of methods known in the art. Examples include the use of an inhaler or nebulizer, formulation with an aerosolizing agent, or via perfusion in a fluorocarbon or synthetic pulmonary surfactant. Compounds may be delivered in the context of a vesicle such as a liposome or any other natural or synthetic vesicle. Additional examples of suitable modes of administration are well known in the art.

nNOS: neuronal nitric acid synthase. This enzyme converts arginine to L-citrulline as shown in FIG. 1. When uncoupled from BH4 (H4 Biopterin) it produces reactive oxygen species which can form peroxynitrite.

nNOS inhibitor. A compound that inhibits the activity of nNOS which may be reflected by its decreased ability to produce reactive oxygen species like peroxynitrite. These include 7-nitroindazole, JI-8 (cis-N¹-[4′-6″-amino-(4″-methylpyridin-2″-ylmethyppyrrolidin-3′-yl]-N²-[2′-(3″-fluorophenypethyl]ethane-1,2-diamine), and HJ619 (cis-N.sup.1-[4′-6″-Amino-4″-methyl-pyridin-2″-ylmethyl)-pyrrolidin-3′-yl]-N.sup.2-(4′-chloro-benzyl)-ethane-1,2-diamine).

Subject. The term “subject” refers to any animal (e.g., a mammal), including, but not limited to, humans, non-human primates, rodents, and the like, which is to be the recipient of a particular treatment. Advantageously, the subject s in utero or a preterm infant having congenital heart disease or at increased risk of having congenital heart disease. This term is inclusive of the mother of a subject in utero. Advantageously, a subject according to the invention is a human foetus in utero having or at risk of having congenital heart disease or a foetus in utero that has or will undergo surgery for congenital heart disease. A broader class of subjects experiencing hypoxia including preterm infants, neonates, toddlers, children and adults may also be treated, especially individuals whose brains or nervous system are developing. A subject may be one at risk of later developing any of the deficits, impairments, behavioural or other problems associated with occurrence of CHD described herein or any of the physiological or circulatory conditions associated with CHD.

A subject to be treated with the method according to the invention need not have a particular enzymatic deficiency, such as a genetic BH4 deficiency (e.g., dopa-responsive dystonia, sepiapterin reductase (SR) deficiency, or dihydropteridine reductase deficiency), elevated phenylalanine levels associated with PKU or decreased tyrosine or tryptophan levels, such as those associated with reduced phenylalanine hydroxylase, tyrosine hydroxylase, or tryptophan hydroxylase activity. Conditions associated with elevated phenylalanine levels include phenylketonuria, both mild and classic, hyperphenylalai inemia and other conditions where phenylalanine is elevated above normal levels. Conditions associated with decreased tyrosine or tryptophan levels include neurotransmitter deficiency, neurological and psychiatric disorders such as Parkinson's, dystonia, spinocerebellar degeneration, pain, fatigue, depression, other affective disorders and schizophrenia. A subject according to the invention does not necessarily include a person having phenylketonuria (PKC), Parkinson's disease, dystonia, depression, Rett Syndrome, infantile autism, senile dementia, Alzheimer's disease or atherosclerosis.

White matter (“WM”). White matter is the paler tissue of the brain and spinal cord, consisting mainly of nerve fibers with their myelin sheaths. The white matter is white because of a fatty substance (myelin) that surrounds the nerve fibers (axons). Myelin is found in almost all long nerve fibers, and acts as electrical insulation increasing the speed of transmission of nerve signals. White matter, long thought to be passive tissue, actively affects how the brain learns and functions. While grey matter is primarily associated with processing and cognition, white matter modulates the distribution of action potentials, acting as a relay and coordinating communication between different brain regions⁶¹. Oligodendrocytes synthesize myelin and the most prominent cell population in white matter. Activation of nitric oxide synthase (NOS) produces reactive oxygen species and peroxynitrite which is toxic to oligodendrocytes during chronic hypoxia⁹. Moreover, tetrahydrobiopterin (BH4) availability is significantly reduced upon activation of NOS and leads to NOS uncoupling and production of reactive oxygen species and toxic peroxynitrite (FIGS. 1 and 2).

Nonlimiting embodiments of the invention include the following.

A method for treating a subject having, or at risk of having, reduced oxygen delivery to, or hypoxia of, the brain or nervous system comprising administering tetrahydrobiopterin (BH4), a structural analog of BH4 having the same two ring heterocyclic core ring structure which may have alternative substituents, another cofactor for nitric oxide synthase, or an inhibitor of nNOS to a subject in need thereof. The subject may be in utero and a pregnant woman, a preterm infant, neonate, child or adult having hypoxia or at risk of having hypoxia in the brain or nervous system, especially, a foetus, preterm infant, or child whose brain is growing or developing and thus is susceptible to disruptions to growth or development associated with hypoxia or subnormal levels of BH4.

A subject may also be one who is at risk, who has been diagnosed to be at risk, or who has complex congenital heart defect or disease, including complex congenital heart disease (CHD). Such a subject may also be one who is at risk, has been diagnosed with, or who has white matter injury or insult or who has been diagnosed with abnormal low levels of myelin basic protein, increased oligodendrocyte density, or increased apoptosis of oligodendrocytes or glial cells in the nervous system compared to a normal or control subject. A subject may also have had, or be at risk of, traumatic injury to the brain or nervous system including diffuse white matter or diffuse axonal injury in the brain or nervous system. A subclass of subjects may have or had had phenylketonuria (PKU), BH4-deficient hyperphenylalanininemia, or a genetic or inherited deficiency of tetrahydrobiopterin (BH4). A subject also, specifically, includes a pregnant woman who carries a subject in need of treatment. A subject is preferably a human; however, the invention also includes treatment of other mammalian or animal subjects who express nNOS or functionally and/or structurally similar enzymes.

Advantageously, the method comprises administering tetrahydrobiopterin (BH4) to the subject, optionally, along with a carrier or excipient. Other active ingredients such as antioxidants or other compounds that affect nNOS activity or affect production of NO or active oxides by nNOS may be coadministered before, at the same time or after the BH4.

Another aspect of the invention is a composition comprising tetrahydrobiopterin (BH4), a structural analog of BH4 having the same two ring heterocyclic core ring structure which may have alternative substituents and that exhibits a similar ability as BH4 to interact with nNOS and reduce hypoxia, a non-BH4 cofactor for nitric oxide synthase, or an inhibitor of nNOS. Such a composition may be formulated for administration to the subject to the subject's mother when the subject is in utero.

Medical devices suitable for single, metered, multiple or continuous administration of compositions according to the invention are also contemplated. A device may be designed or adapted to control, meter, measure a precise volume or amount of BH4 or other composition according to the invention to a subject. It may also be designed or adapted to administer BH4 to a subject in utero, to a subject at risk of hypoxia or undergoing hypoxia, or to a subject undergoing a surgical procedure, such as correction of CHD or complex CHD. Such devices or compositions may be included in a kit that includes packaging materials or instructions for use.

The following Examples describe certain, nonlimiting features of the invention.

### EXAMPLES

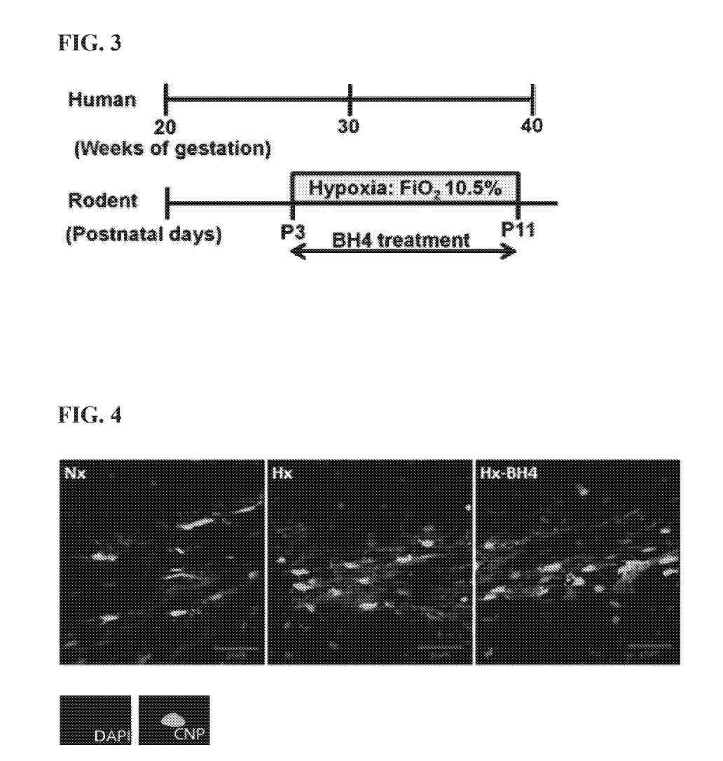

Three groups of mice: Normoxic controls (Nx), Hypoxic (Hx), Hypoxic treated with BH4 (Hx-BH4). Hx and Hx-BH4 mice were kept at 10.5% FiO₂ from postnatal day 3 to 11—a period of WM development equivalent to the 3^{rd} trimester in humans as illustrated by FIG. 3.

BH4 brain levels were quantified and compared between Nx (n=11) and Hx (n=12). Densities of cells expressing CNP, a marker of oligodendrocytes which are cells responsible for myelination, and Caspase3, an apoptosis marker, were compared between all groups (n=3-6 each). Expression of myelin basic protean, a myelination marker, was quantified using Western Blot (n=7-12 each).

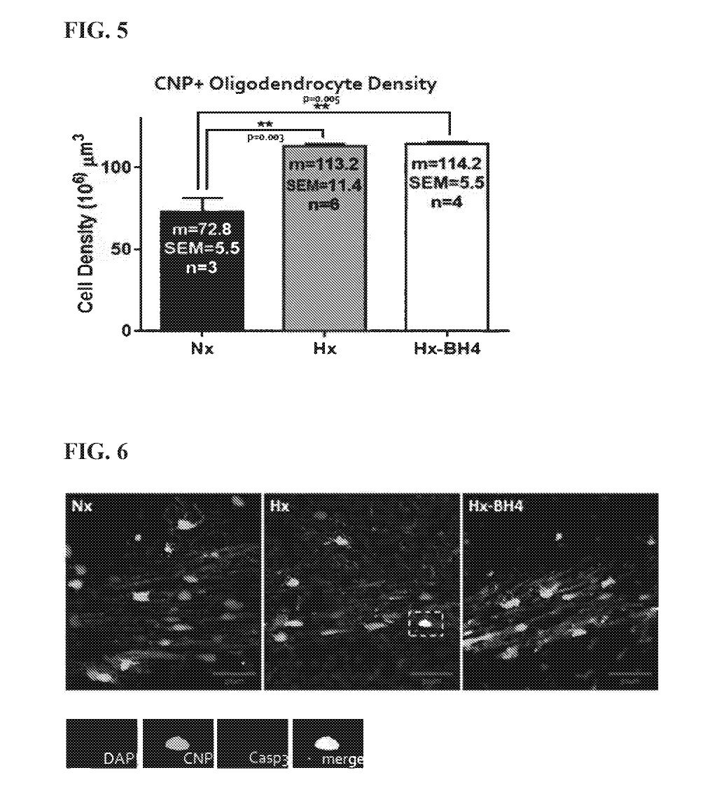

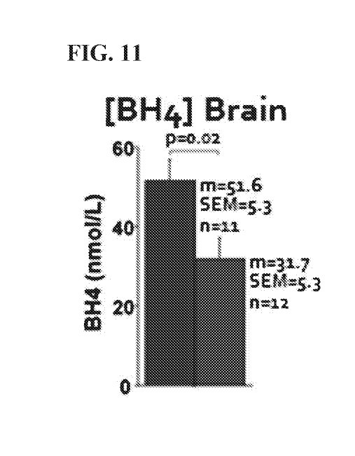

As shown by FIG. 11, brain BH4 levels were 38.4% lower in Hx compared to Nx 0.02). Oligodendrocytes were increased in Hx compared to Nx (p=0.003), which is consistent with hypoxia-induced proliferation and BH4 treatment did not limit this effect, see FIGS. 4 and 5.

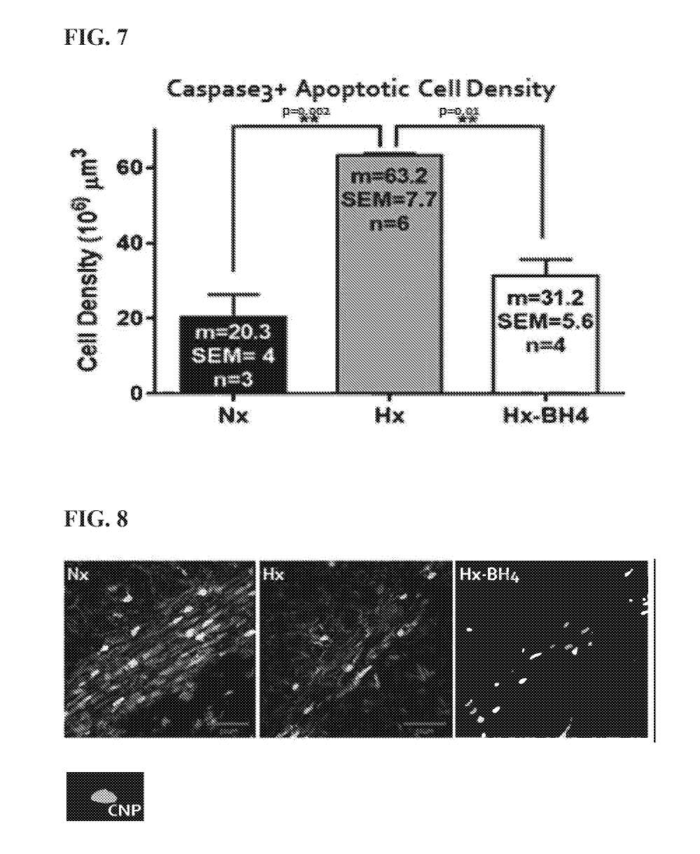

Hx showed increased WM apoptosis (p=0.002), which decreased with BH4 treatment (p=0.01). Remarkably, there was no difference in apoptotic cell density between Nx and Hx-BH4, see FIGS. 6 and 7.

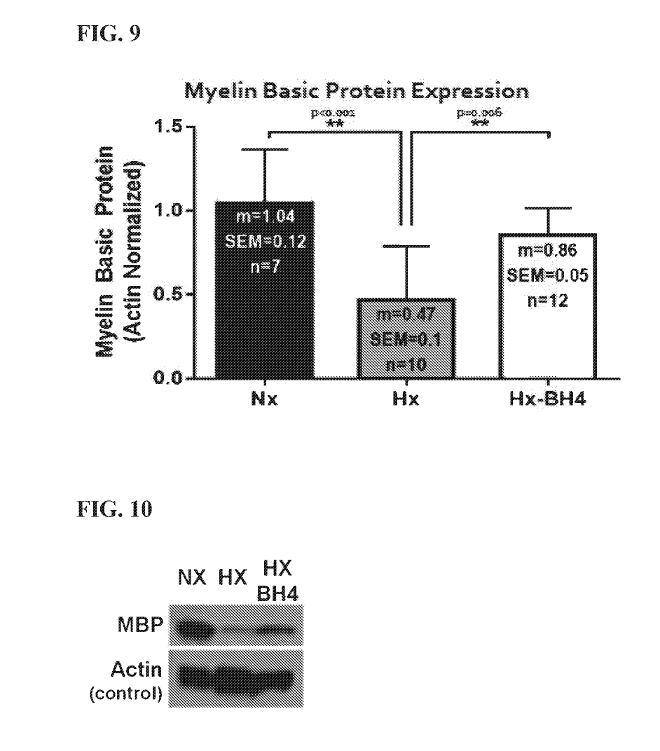

Hx showed decreased expression of myelin basic protein (p<0.001), which increased with BH4 treatment (p=0.006). Again, there was no difference between Nx and Hx-BH4; FIGS. 8, 9 and 10.

These results are consistent with depletion by hypoxia of brain BH4 levels, increased white matter apoptosis and loss of myelination due to hypoxia, and the surprising finding that hypoxic effects were mitigated by treated with BH4. A reduction in rain BH4 levels were depleted in hypoxia.

The foregoing description discloses particular embodiments. As will be understood by those skilled in the art, the approaches, methods, techniques, materials, devices, and so forth disclosed herein may be embodied in additional embodiments as understood by those of skill in the art, it is the intention of this application to encompass and include such variation. Accordingly, this description is illustrative and should not be taken as limiting the scope of the following claims.

Citations herein are incorporated by reference especially for the purposes of specifically describing the referenced subject matter or the subject matter in the same or adjoining paragraphs or sections.

### CITED REFERENCES

1. Marelli A J, Mackie A S, Ionescu-Ittu R, Rahme E and Pilote L. Congenital heart disease in the general population: changing prevalence and age distribution. Circulation. 2007; 115:163-72.

2. Lenfant C. Report of the Task Force on Research in Pediatric Cardiovascular Disease. Circulation. 2002; 106:1037-42.

3. Hoffman J. The global burden of congenital heart disease. Cardiovascular journal of Africa. 2013; 24:141-5.

4. Botto L D, Correa A and Erickson J D. Racial and temporal variations in the prevalence of heart defects. Pediatrics. 2001; 107:E32.

5. Al-Radi O O, Harrell F E, Jr., Caldarone C A, McCrindle B W, Jacobs J P, Williams M G, Van Arsdell G S and Williams W G. Case complexity scores in congenital heart surgery: a comparative study of the Aristotle Basic Complexity score and the Risk Adjustment in Congenital Heart Surgery (RACHS-1,) system. J Thorac Cardiovasc Surg. 2007; 133:865-75.

6. Bellinger D C, Jonas R A, Rappaport L A, Wypij D, Wernovsky G, Kuban K C, Barnes P D, Holmes G L, Hickey P R, Strand R D and et al. Developmental and neurologic status of children after heart surgery with hypothermic circulatory arrest or low-flow cardiopulmonary bypass. N Engl J Med. 1995; 332:549-55.

7. Bellinger D C, Wypij D, Kuban K C, Rappaport L A, Hickey P R, Wernovsky G, Jonas R A and Newburger J W. Developmental and neurological status of children at 4 years of age after heart surgery with hypothermic circulatory arrest or low-flow cardiopulmonary bypass. Circulation. 1999; 100:526-32.

8. Bellinger D C, Wypij D, Rivkin M J, Demaso D R, Robertson R L, Dunbar-Masterson C, Rappaport L A, Wernovsky G, Jonas R A and Newburger J W. Adolescents With d-Transposition of the Great Arteries Corrected With the Arterial Switch Procedure: Neuropsychological Assessment and Structural Brain Imaging. Circulation. 2011.

9. Majnemer A, Limperopoulos C, Shevell M, Rohlicek C, Rosenblatt B and Tchervenkov C. Developmental and functional outcomes at school entry in children with congenital heart defects. J Pediatr. 2008; 153:55-60.

10. Wernovsky G. Current insights regarding neurological and developmental abnormalities in children and young adults with complex congenital cardiac disease. Cardiol Young. 2006; 16 Suppl 1:92-104.

11. Tabbutt S, Gaynor J W and Newburger J W. Neurodevelopmental outcomes after congenital heart surgery and strategies for improvement. Curr Opin Cardiol. 2012; 27:82-91. 33

12. Newburger J W, Sleeper L A, Bellinger D C, Goldberg C S, Tabbutt S, Lu M, Mussatto K A, Williams I A, Gustafson K E, Mital S, Pike N, Sood E, Mahle W T, Cooper D S, Dunbar-Masterson C, Krawczeski C D, Lewis A, Merron S C, Pemberton V L, Ravishankar C, Atz T W, Ohye R G and Gaynor J W. Early Developmental Outcome in Children with Hypoplastic Left Heart Syndrome and Related Anomalies: The Single Ventricle Reconstruction Trial. Circulation. 2012.

13. Marelli A J, Ionescu-Ittu R, Mackie A S, Guo L, Dendukuri N and Kaouache M. Lifetime prevalence of congenital heart disease in the general population from 2000 to 2010. Circulation. 2014; 130:749-56.

14. Oster M E, Lee K A, Honein M A, Riehle-Colarusso Shin M and Correa A. Temporal trends in survival among infants with critical congenital heart defects. Pediatrics. 2013; 131:e1502-8.

15. Kaltman J R, Andropoulos D B, Checchia P A, Gaynor J W, Hoffman T M, Laussen P C, Ohye R G, Pearson G D, Pigula F, Tweddell J, Wernovsky G and Del Nido P. Report of the pediatric heart network and national heart, lung, and blood institute working group on the perioperative management of congenital heart disease. Circulation. 2010; 121:2766-72.

16. Newburger J W, Sleeper L A, Bellinger D C, Goldberg C S, Tabbutt S, Lu M, Mussatto K A, Williams I A, Gustafson K E, Mital S, Pike N, Sood E, Mahle W T, Cooper D S, Dunbar-Masterson C, Krawczeski C D, Lewis A, Menon S C, Pemberton V L, Ravishankar C, Atz T W, Ohye R G, Gaynor J W and Pediatric Heart Network I. Early developmental outcome in children with hypoplastic left heart syndrome and related anomalies: the single ventricle reconstruction trial. Circulation. 2012; 125:2081-91.

17. Goldberg C S, Lu M, Sleeper L A, Mahle W T, Gaynor J W, Williams I A, Mussatto K A, Ohye R G, Graham E M, Frank D U, Jacobs J P, Krawczeski C, Lambert L, Lewis A, Pemberton V L, Sananes R, Sood B, Wechsler S B, Bellinger D C, Newburger J W and Pediatric Heart Network I. Factors associated with neurodevelopment for children with single ventricle lesions. J Pediatr. 2014; 165:490-496 e8.

18. Andropoulos D B, Easley R B, Brady K, McKenzie E D, Heinle J S, Dickerson H A, Shekerdemian L S, Meador M, Eiseman C, Hunter J V, Turcich M, Voigt R G and Fraser C D, Jr. Neurodevelopmental outcomes after regional cerebral perfusion with neuromonitoring for neonatal aortic arch reconstruction. Ann Thorac Surg. 2013; 95:648-54; discussion 654-5. 40

19. Volpe J J. Hypoxic-ischemic enchephalopathy: Biochemical and Physiological aspects. 5th ed. Philadelphia: Saunders Elsevier; 2008.

20. Marino B S, Lipkin P H, Newburger J W, Peacock G, Gerdes M, Gaynor J W, Mussatto K A, Uzark K, Goldberg C S, Johnson W H, Jr., Li J, Smith S E, Bellinger D C and Mahle W T. Neurodevelopmental Outcomes in Children With Congenital Heart Disease: Evaluation and Management: A Scientific Statement From the American Heart Association. Circulation. 2012; 126:1143-1172.

21. Gaynor J W, Stopp C, Wypij D, Andropoulos D B, Atallah J, Atz A M, Beca J, Donofrio M T, Duncan K, Ghanayem N S, Goldberg C S, Hovels-Gurich H, Ichida F, Jacobs J P, Justo R, Latal B, Li J S, Mahle W T, McQuillen P S, Menon S C, Pemberton V L, Pike N A, Pizarro C, Shekerdemian L S, Synnes A, Williams I, Bellinger D C, Newburger J W and International Cardiac Collaborative on Neurodevelopment I. Neurodevelopmental outcomes after cardiac surgery in infancy. Pediatrics. 2015; 135:816-25.

22. Back S A, Riddle A and McClure M M. Maturation-dependent vulnerability of perinatal white matter in premature birth. Stroke. 2007; 38:724-30. 42

23. Salmaso N, Jablonska B, Scafidi J, Vaccarino F M and Gallo V. Neurobiology of premature brain injury. Nat Neurosci. 2014; 17:341-6.

24. Bellinger D C, Wypij D, Rivkin M J, DeMaso D R, Robertson R L, Jr., Dunbar-Masterson C, Rappaport L A, Wernovsky G, Jonas R A and Newburger J W. Adolescents with d-transposition of the great arteries corrected with the arterial switch procedure: neuropsychological assessment and structural brain imaging. Circulation. 2011; 124:1361-9.

25. DeMaso D R, Labella M, Taylor G A, Forbes P W, Stopp C, Bellinger D C, Rivkin M J, Wypij D and Newburger J W. Psychiatric Disorders and Function in Adolescents with d-Transposition of the Great Arteries. J Pediatr. 2014; 165:760-6.

26. Rivkin M J, Watson C G, Scoppettuolo L A, Wypij D, Vajapeyam S, Bellinger D C, Demaso D R, Robertson R L, Jr. and Newburger J W. Adolescents with d-transposition of the great arteries repaired in early infancy demonstrate reduced white matter microstructure associated with clinical risk factors. J Thorac Cardiovasc Surg. 2013; 146:543-549 e1.

27. Rollins C K, Watson C G, Asaro L A, Wypij D, Vajapeyam S, Bellinger D C, DeMaso D R, Robertson R L, Jr., Newburger J W and Rivkin M J. White matter microstructure and cognition in adolescents with congenital heart disease. J Pediatr. 2014; 165:936-44 e1-2.

28. Panigrahy A, Schmithorst V J, Wisnowski J L, Watson C G, Bellinger D C, Newburger J W and Rivkin M J. Relationship of white matter network topology and cognitive outcome in adolescents with d-transposition of the great arteries. NeuroImage Clinical. 2015; 7:438-48,

29. Ment L R, Hirtz D and Huppi P S. Imaging biomarkers of outcome in the developing preterm brain. Lancet Neurol. 2009; 8:1042-55.

30. Donofrio M T, Duplessis A J and Limperopoulos C. Impact of congenital heart disease on fetal brain development and injury. Curr Opin Pediatr. 2011; 23:502-11.

31. McQuillen P S and Miller S P. Congenital heart disease and brain development. Ann N Y Acad Sci. 2010; 1184:68-86.

32. Sun L, Macgowan C K, Sled J G, Yoo S J, Manlhiot C, Porayette P, Grosse-Wortmann L, Jaeggi E, McCrindle B W, Kingdom J, Hickey E, Miller S and Seed M. Reduced fetal cerebral oxygen consumption is associated with smaller brain size in fetuses with congenital heart disease. Circulation. 2015; 131:1313-23.

33. Miller S P, McQuillen P S, Hamrick S, Xu D, Glidden D V, Charlton N, Karl T, Azakie A, Ferriero D M, Barkovich A J and Vigneron D B. Abnornal brain development in newborns with congenital heart disease. N Engl Med. 2007; 357:1928-38.

34. Limperopoulos C, Tworetzky W, McElhinney D B, Newburger J W, Brown D W, Robertson R L, Jr., Guizard N, McGrath E, Geva J, Annese D, Dunbar-Masterson C, Trainor B, Laussen P C and du Plessis A J. Brain volume and metabolism in fetuses with congenital heart disease: evaluation with quantitative magnetic resonance imaging and spectroscopy. Circulation. 2010; 121:26-33.

35. Licht D J, Shera D M, Clancy R R, Wernovsky G, Montenegro L M, Nicolson S C, Zimmerman R A, Spray T L, Gaynor J W and Vossough A. Brain maturation is delayed in infants with complex congenital heart defects. J Thorac Cardiovasc Surg. 2009; 137:529-36; discussion 536-7.

36. Clouchoux C, du Plessis A J, Bouyssi-Kobar M, Tworetzky W, McElhinney D B, Brown D W, Gholipour A, Kudelski D, Warfield S K, McCarter R J, Robertson R L, Jr., Evans A C, Newburger J W and Limperopoulos C. Delayed Cortical Development in Fetuses with Complex Congenital Heart Disease. Cereb Cortex. 2012.

37. Andropoulos D B, Hunter S V, Nelson D P, Stayer S A, Stark A R, McKenzie E D, Heinle J S, Graves D E and Fraser C D, Jr. Brain immaturity is associated with brain injury before and after neonatal cardiac surgery with high-flow bypass and cerebral oxygenation monitoring. J Thorac Cardiovasc Surg. 2010; 139:543-56.

38. Beca J, Gunn J K, Coleman L, Hope A, Reed P W, Hunt R W, Finucane K, Brizard C, Dance B and Shekerdemian L S. New white matter brain injury after infant heart surgery is associated with diagnostic group and the use of circulatory arrest. Circulation. 2013; 127:971-9. 8

39. Morton P D, Ishibashi N, Jonas R A and Gallo V. Congenital cardiac anomalies and white matter injury. Trends Neurosci. 2015; 38:353-363. 4

40. White D A, Antenor-Dorsey J A, Grange D K, Hershey T, Rutlin J, Shimony J S, tvicKinstry R C and Christ S E. White matter integrity and executive abilities following treatment with tetrahydrobiopterin (BH4) in individuals with phenylketonuria. Molecular genetics and metabolism. 2013; 110:213-7.

41. Peng H, Peck D, White D A and Christ S E. Tract-based evaluation of white matter damage in individuals with early-treated phenylketonuria. Journal of inherited metabolic disease. 2014; 37:237-43.

42. Taylor D L, Edwards A D and Mehmet H. Oxidative metabolism, apoptosis and perinatal brain injury. Brain Pathol. 1999; 9:93-117.

43. Silverstein F S and Jensen F E. Neonatal seizures. Ann Neurol. 2007; 62:112-20. 55

44. Blomgren K and Hagberg H. Free radicals, mitochondria, and hypoxia-ischemia in the developing brain. Free Radic Biol Med. 2006; 40:388-97.

45. Robertson C L. Scafidi S, McKenna M C and Fiskum G. Mitochondrial mechanisms of cell death and neuroprotection in pediatric ischemic and traumatic brain injury. Exp Neurol. 2009; 218:371-80.

46. Berka V, Wang L H and Tsai A L. Oxygen-induced radical intermediates in the nNOS oxygenase domain regulated by L-arginine, tetrahydrobiopterin, and thiol. Biochemistry. 2008; 47:405-20.

47. Scafidi J and Gallo V. New concepts in perinatal hypoxia ischemia encephalopathy. Curr Neurol Neurosci Rep. 2008; 8:130-8.

48. Raymond M, Li P, Mangin J M, Huntsman M and Gallo V. Chronic Perinatal Hypoxia Reduces Glutamate-Aspartate Transporter Function in Astrocytes through the Janus Kinase/Signal Transducer and Activator of Transcription Pathway. J Neurosci. 2011; 31:17864-71.

49. Administration FaD. FDA labelling information [online], <http://www.fda.gov/cder/foi/label/2007/022181Ibl.pdf>.2007.

50. Levy H L, Milanowski A, Chakrapani A, Cleary M, Lee P, Trefz F K, Whitley C B, Feillet F, Feigenbaum A S, Bebchuk J D, Christ-Schmidt H, Dorenbaum A and Sapropterin Research G. Efficacy of sapropterin dihydrochloride (tetrahydrobiopterin, 6R-BH4) for reduction of phenylalanine concentration in patients with phenylketonuria: a phase III randomised placebo-controlled study. Lancet. 2007; 370:504-10.

51. Trefz F K, Burton B K, Longo N, Casanova M M, Gruskin D J, Dorenbaum A, Kakkis E D, Crombez E A, Grange D K, Harmatz P, Lipson M H, Milanowski A, Randolph L M, Vockley J, Whitley C B, Wolff J A, Bebchuk J, Christ-Schmidt H, Hermermann J B and Sapropterin Study G. Efficacy of sapropterin dihydrochloride in increasing phenylalanine tolerance in children with phenylketonuria: a phase III, randomized, double-blind, placebo-controlled study. J Pediatr. 2009; 154:700-7.

52. Burton B K, Nowacka M, Hennermann J B, Lipson M, Grange D K, Chakrapani A, Trefz F, Dorenbaum A, Imperiale M, Kim S S and Fernhoff P M. Safety of extended treatment with sapropterin dihydrochloride in patients with phenylketonuria: results of a phase 3b study. Molecular genetics and metabolism. 2011; 103:315-22.

53. Gizewska M, Hnatyszyn G, Sagan L, Cyrylowski L, Zekanowski C, Modrzejewska M, Nestorowicz B, Kubalska J and Walczak M. Maternal tetrahydrobiopterin deficiency: the course of two pregnancies and follow-up of two children in a mother with 6-pyruvoyl-tetrahydropterin synthase deficiency. Journal of inherited metabolic disease. 2009; 32 Suppl 1:S83-9.

54. Grange D K, Hillman R E, Burton B K, Yano S, Vockley J, Fong C T, Hunt J, Mahoney J J, Cohen-Pfeffer J L, Phenylketonuria Demographics O, Safety r and Maternal Phenylketonuria Observational Program s-r. Sapropterin dihydrochloride use in pregnant women with phenylketonuria: An interim report of the PKU MOMS sub-registry. Molecular genetics and metabolism. 2014; 112:9-16.

55. Feillet F, Muntau A C, Debray F G, Lotz-Havla A S, Puchwein-Schwepcke A, Fofou-Caillierez M B, van Spronsen F and Trefz F F. Use of sapropterin dihydrochloride in maternal phenylketonuria. A European experience of eight cases. Journal of inherited metabolic disease. 2014.

56. Trefz F K, Muntau A C, Lagler F B, Moreau F, Alm J, Burlina A, Rutsch F, Belanger-Quintana A, Feillet F and investigators K. The Kuvan((R)) Adult Maternal Paediatric European Registry (KAMPER) Multinational Observational Study: Baseline and 1-Year Data in Phenylketonuria Patients Responsive to Sapropterin. JIMD Rep. 2015; 23:35-43.

57. Vasquez-Vivar J, Whitsett J, Derrick M, Ji X, Yu L and Tan S. Tetrahydrobiopterin in the prevention of hypertonia in hypoxic fetal brain. Ann Neurol. 2009; 66:323-31.

58. Ishibashi N, Scafidi J, Murata A, Korotcova L, Zurakowski D, Gallo V and Jonas R A. White matter protection in congenital heart surgery. Circulation, 2012; 125:859-71.

59. Murata A, Agematsu K, Korotcova L, Gallo V, Jonas R A and Ishibashi N. Rodent brain slice model for the study of white matter injury. J Thorac Cardiovasc Surg. 2013; 146:1526-1533 e1.

60. Agematsu K, Korotcova L, Scafidi J, Gallo V, Jonas R A and Ishibashi N. Effects of preoperative hypoxia on white matter injury associated with cardiopulmonary bypass in a rodent hypoxic and brain slice model. Pediatr Res. 2014; 75:618-25.

61. Fields, D. R. (2008). “White Matter Matters”. Scientific American. 298 (3): 54-61.

1. A method for treating a subject having, or at risk of having, reduced oxygen delivery to, or hypoxia of, the brain or nervous system comprising administering tetrahydrobiopterin (BH4) to said subject.

2. The method of claim 1, wherein the subject is in utero.

3. The method of claim 1, wherein the subject is a preterminfant.

4. The method of claim 1 wherein the subject is a neonate.

5. The method of claim 1, wherein the subject is a child or an adult.

6. The method of claim 1, wherein the subject is at risk of having complex congenital heart disease.

7. The method of claim 1, wherein the subject has been diagnosed with complex congenital heart disease (CHD).

8. The method of claim 1, wherein the subject has been diagnosed with white matter injury.

9. The method of claim 1, wherein the subject has been diagnosed with abnormal low levels of myelin basic protein, increased oligodendrocyte density, or increased, or risk of, apoptosis of oligodendrocytes in the nervous system compared to a normal or control subject.

10. The method of claim 1, wherein the subject has had traumatic injury to the brain or nervous system

11. The method of claim 1, wherein the subject has diffuse white matter or diffuse axonal injury in the brain or nervous system.

12. The method of claim 1, wherein said subject does not have phenylketonuria (PKU), BH4-deficient hyperphenylalanininemia, or a genetic or inherited deficiency of tetrahydrobiopterin (BH4).

13. The method of claim 1 that comprises administering tetrahydrobiopterin (BH4) to the mother of the subject.

14. The method of claim 1 that comprises administering tetrahydrobiopterin (BH4) to the subject.

15. The method of claim 1, wherein the subject is a mammal.

16. The method of claim 1, wherein the subject is human.

17. A composition comprising tetrahydrobiopterin (BH4) in a form suitable for administration to a subject in utero having complex congenital heart disease.

18. The composition of claim 17, further comprising arginine or at least one other ingredient that enhances NO production.

19. The composition of claim 17 further comprising arginine of at least one other ingredient that nNOS inhibitor, antioxidant, or other ingredient that inhibits the production or that reduces the concentration of reactive oxides.

20. A medical device suitable for administering the composition of claim 17 to a subject in utero having complex heart disease.
